SEQUENCE LISTING (1 sequence)

SEQ ID NO: 1 - DNA, 100 bp, Artificial Sequence
  Description of Artificial Sequence Ordering of Nucleotide Species Flows
tacgtacgtctgagcatcgatcgatgtacagctacgtacgtctgagcatcgatcgatgtacagctacgtacgtctgagcatcgatcgatgtacagctacg

(19) United States
(12) Patent Application Publication
(10) Pub. No.: US 2019/0237163 A1
(43) Pub. Date: Aug. 1, 2019

(54) METHODS FOR FLOW SPACE QUALITY SCORE PREDICTION BY NEURAL NETWORKS

(71) Applicant: LIFE TECHNOLOGIES CORPORATION, Carlsbad, CA (US)

(72) Inventors: Chao Wang, Santa Clara, CA (US); Eugene INGERMAN, San Francisco, CA (US)

(21) Appl. No.: 16/245,343

(22) Filed: Jan. 11, 2019

Related U.S. Application Data
(60) Provisional application No. 62/617,101, filed on Jan. 12, 2018.

(51) Int. Cl. G16B 40/10; G16B 30/00; G06N 3/08

(52) U.S. Cl. CPC G16B 40/10; G06N 3/08; G16B 30/00

(57) ABSTRACT

An artificial neural network is applied to a plurality of flow predictor features to generate a flow space probability of error for a base call. A base quality value for the base call is determined based on the flow space probability of error. The base call and flow predictor features are based on the flow space signal measurements generated in response to the nucleotide flow to the reaction confinement region. For an array of reaction confinement regions, a plurality of parallel neural networks is applied to produce a probability of error for each reaction confinement region. A given neural network of the parallel neural networks is applied to the plurality of flow predictor features corresponding to a given reaction confinement region in the array to provide the flow space probability of error for the given reaction confinement region.

### CROSS-REFERENCE TO RELATED APPLICATIONS

This application claims benefit under 35 U.S.C. 119 to U.S. application Ser. No. 62/617,101, filed on Jan. 12, 2018. The entire content of the aforementioned application is incorporated by reference herein.

### BACKGROUND

Various instruments, apparatuses, and/or systems for sequencing nucleic acids sequence nucleic acids using sequencing-by-synthesis. Such instruments, apparatuses, and/or systems may include, for example, the Genome Analyzer/HiSeq/MiSeq platforms (Illumina, Inc.; see, e.g.., U.S. Pat. Nos. 6,833,246 and 5,750,341); the GS FLX, GS FLX Titanium, and GS Junior platforms (Roche/454 Life Sciences; see, e.g., Ronaghi et al., SCIENCE, 281:363-365 (1998), and Margulies et al., NATURE, 437:376-380 (2005)); and the Ion Personal Genome Machine (PGM™), Ion Proton™ and Ion S5™ (Life Technologies Corp./Ion Torrent; see, e.g., U.S. Pat. No. 7,948,015 and U.S. Pat. Appl. Publ. Nos. 2010/0137143, 2009/0026082, and 2010/0282617, which are all incorporated by reference herein in their entirety).

As part of the output, such systems are expected to produce a Phred Quality Score (Brent Ewing, LaDeana W. Hillier, Michael C. Wendl, Phil Green; Base-calling of automated sequencer traces using Phred. I. accuracy assessment. Genome Research, Issue: 3, Volume: 8, Pages: 175-185. Feb. 28, 1998) for each base of the identified sequence. Phred Quality Score is proportional to the logarithm of base-calling error probability and is based on the measurements of the signal quantities specific to each type of NGS instrument during sequencing. For known DNA samples, the Phred Quality Score is expected to match closely a posteriori error measurements (based on aligning the sequence produced by the instrument with the known sample sequence).

As part of generating base sequence, NGS systems identify and remove from output, parts of base call sequence with low fidelity. For Ion instruments, such an identification is based on the Phred Quality Score. Thus, accurate Phred Quality Score is important for producing the largest possible number of high fidelity bases.

### SUMMARY

According to an exemplary embodiment, there is provided a method for estimating quality values of nucleotide base calls, comprising: (a) receiving flow space signal measurements from a reaction confinement region, the flow space signal measurements generated in response to a nucleotide flow to the reaction confinement region in an array of reaction confinement regions; (b) generating a base call and a plurality of flow predictor features corresponding to the nucleotide flow based on the flow space signal measurements; (c) applying an artificial neural network to the plurality of flow predictor features to generate a flow space probability of error; and (d) determining a base quality value based on the flow space probability of error.

According to an exemplary embodiment, there is provided a system for estimating quality values of nucleotide base calls, comprising a machine-readable memory and a processor configured to execute machine-readable instructions, which, when executed by the processor, cause the system to perform a method for compressing molecular tagged nucleic acid sequence data, comprising: (a) receiving, at the processor, flow space signal measurements from a reaction confinement region, the flow space signal measurements generated in response to a nucleotide flow to the reaction confinement region in an array of reaction confinement regions; (b) generating a base call and a plurality of flow predictor features corresponding to the nucleotide flow based on the flow space signal measurements; (c) applying an artificial neural network to the plurality of flow predictor features to generate a flow space probability of error; and (d) determining a base quality value based on the flow space probability of error.

According to an exemplary embodiment, there is provided a non-transitory machine-readable storage medium comprising instructions which, when executed by a processor, cause the processor to perform a method for estimating quality values of nucleotide base calls, comprising: (a) receiving, at the processor, flow space signal measurements from a reaction confinement region, the flow space signal measurements generated in response to a nucleotide flow to the reaction confinement region in an array of reaction confinement regions; (b) generating a base call and a plurality of flow predictor features corresponding to the nucleotide flow based on the flow space signal measurements; (c) applying an artificial neural network to the plurality of flow predictor features to generate a flow space probability of error; and (d) determining a base quality value based on the flow space probability of error.

### BRIEF DESCRIPTION OF THE DRAWINGS

To easily identify the discussion of any particular element or act, the most significant digit or digits in a reference number refer to the figure number in which that element is first introduced.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a flow space quality score prediction system 100 in accordance with one embodiment.

FIG. 2 illustrates a system for nucleic acid sequencing 200 in accordance with one embodiment.

FIG. 3 illustrates a flow cell 300 in accordance with one embodiment.

FIG. 4 illustrates a uniform flow front between successive reagents moving across a section in accordance with one embodiment.

FIG. 5 illustrates a flow cell 300 in accordance with one embodiment.

FIG. 6 illustrates an array section 600 in accordance with one embodiment.

FIG. 7 illustrates a process 700 in accordance with one embodiment.

FIG. 8 shows an exemplary representation of flow space signal measurements from which base calls may be made.

FIG. 9 illustrates a system 904 in accordance with one embodiment.

FIG. 10 illustrates flow predictor parameters 1000 in accordance with one embodiment.

FIG. 11 illustrates a cross-entropy comparisons 1100 in accordance with one embodiment.

FIG. 12 illustrates confusion matrices 1200 in accordance with one embodiment.

FIG. 13 illustrates an example of a basic deep neural network 1300 in accordance with one embodiment.

FIG. 14 illustrates an example of an artificial neuron 1400 in accordance with one embodiment.

FIG. 15 illustrates an example of a quality score system 1500 in accordance with one embodiment.

FIG. 16 illustrates a method 1600 in accordance with one embodiment.

FIG. 17 is an example block diagram of a computing device 1700 that may incorporate embodiments of the present invention.

### DETAILED DESCRIPTION

In this application, “reaction confinement region” generally refers to any region in which a reaction may be confined and includes, for example, a “reaction chamber,” a “well,” and a “microwell” (each of which may be used interchangeably). A reaction confinement region may include a region in which a physical or chemical attribute of a solid substrate can permit the localization of a reaction of interest, and a discrete region of a surface of a substrate that can specifically bind an analyte of interest (such as a discrete region with oligonucleotides or antibodies covalently linked to such surface), for example. Reaction confinement regions may be hollow or have well-defined shapes and volumes, which may be manufactured into a substrate. These latter types of reaction confinement regions are referred to herein as microwells or reaction chambers, and may be fabricated using any suitable microfabrication techniques. Reaction confinement regions may also be substantially flat areas on a substrate without wells, for example.

A plurality of defined spaces or reaction confinement regions may be arranged in an array, and each defined space or reaction confinement regions may be in electrical communication with at least one sensor to allow detection or measurement of one or more detectable or measurable parameter or characteristics. This array is referred to herein as a sensor array. The sensors may convert changes in the presence, concentration, or amounts of reaction by-products (or changes in ionic character of reactants) into an output signal, which may be registered electronically, for example, as a change in a voltage level or a current level which, in turn, may be processed to extract information about a chemical reaction or desired association event, for example, a nucleotide incorporation event. The sensors may include at least one chemically sensitive field effect transistor (“chemFET”) that can be configured to generate at least one output signal related to a property of a chemical reaction or target analyte of interest in proximity thereof. Such properties can include concentration (or a change in concentration) of a reactant, product or by-product, or value of a physical property (or a change in such value), such as ion concentration.

An initial measurement or interrogation of a pH for a defined space or reaction confinement regions, for example, may be represented as an electrical signal or a voltage, which may be digitalized (e.g., converted to a digital representation of the electrical signal or the voltage). Any of these measurements and representations may be considered raw data or a raw signal.

In various embodiments, the phrase “base space” refers to a representation of the sequence of nucleotides. The phrase “flow space” refers to a representation of the incorporation event or non-incorporation event for a particular nucleotide flow. For example, flow space can be a series of values representing a nucleotide incorporation event (such as a one, “1”) or a non-incorporation event (such as a zero, “0”) for that particular nucleotide flow. Nucleotide flows having a non-incorporation event can be referred to as empty flows, and nucleotide flows having at least one nucleotide incorporation event can be referred to as positive flows. It should be understood that zeros and ones are convenient representations of a non-incorporation events and a nucleotide incorporation events; however, any other symbol or designation could be used alternatively to represent and/or identify these events and non-events. In particular, when multiple nucleotides are incorporated at a given position, such as for a homopolymer stretch, the value can be proportional to the number of nucleotide incorporation events and thus the length of the homopolymer stretch.

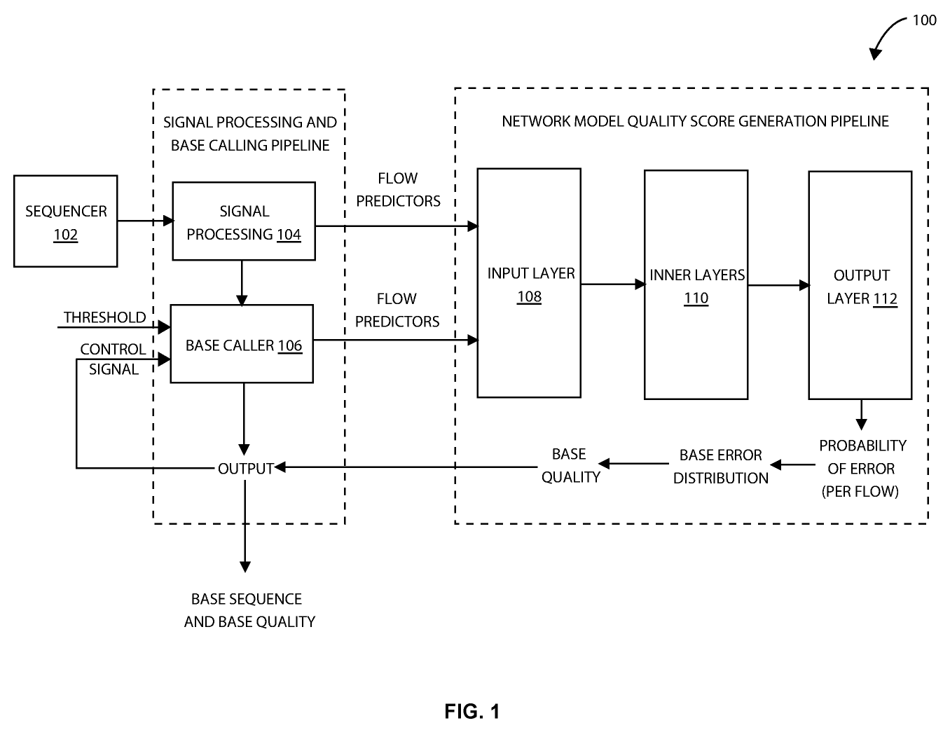

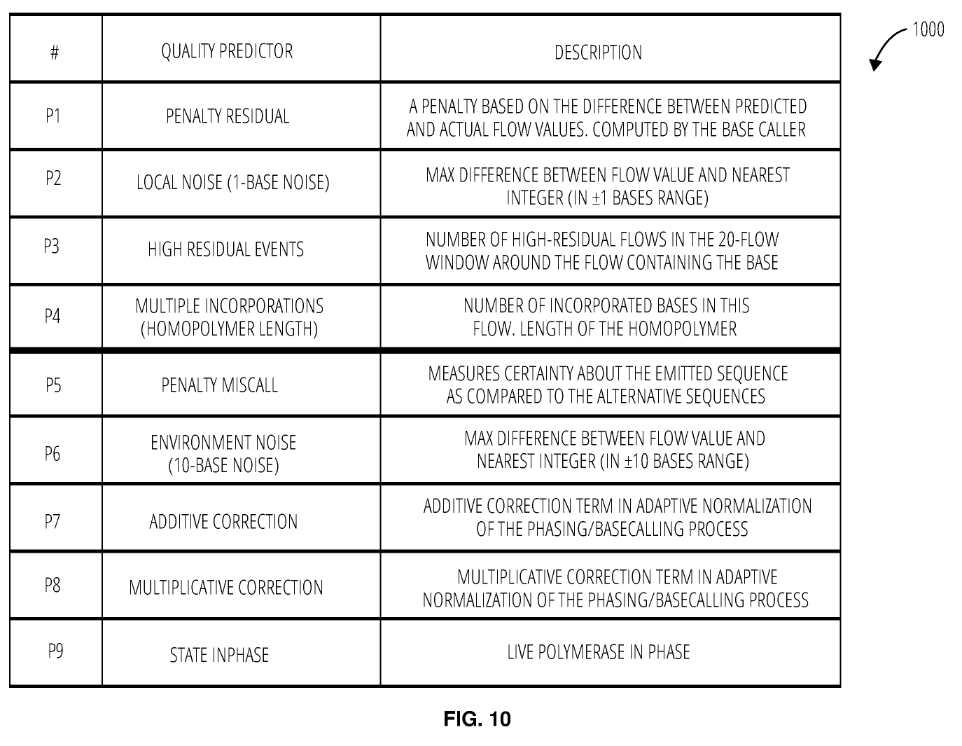

FIG. 1 shows a block diagram for flow space quality score prediction using an artificial neural network model, in accordance with an embodiment. The flow space quality score prediction system 100 comprises a sequencer 102, a signal processing 104, a base caller 106, an input layer 108, inner layers 110, and an output layer 112. The signal processing 104 receives signal data, such as from signal detection unit of a nucleic acid sequencing device (the sequencer 102). The signal data, or flow space signal measurements, are generated in response to nucleotide flow. The signal processing and base calling pipeline provide flow predictor features to the input layer 108 of the neural network. In some embodiments, the flow predictor features can be arranged as one feature vector generated per flow. In some embodiments, the feature vector can include the flow predictor parameters listed in the table of FIG. 10. In some embodiments, the feature vector may include additional, fewer or different parameters.

The input layer 108 may provide various preprocessing functions to the input feature vectors from signal processing 104 and base caller 106. For example, the features may be normalized to fall within a specific range of values. The inner layers 110 shown in FIG. 1 may include one or more layers of processing nodes, or neurons. The number of inner layers and processing nodes per inner layer may be configurable. For example, the number of inner layers can vary from 1 to 10. The number of processing nodes in each layer may also be configurable. For example, the number of nodes (neurons) at a given layer may be in the range of 10 to 256 nodes. For example, the number of nodes at a first inner layer may be 256, at a second inner layer may be 100 and a third inner layer may be 50. In some embodiments, each processing node computes a dot product of the vector of inputs to the node with a weight vector followed by a nonlinear function. In some embodiments, a bias may be added to the dot product prior to applying the nonlinear function, such as rectified linear unit (ReLU). In some embodiments, the nonlinear function includes a sigmoidal function, where z is the result of the dot product:

$$sigmoid ( z ) = \frac{1}{1 + {e}^{- z}} \qquad Equation 1$$

The output of the nonlinear function for each node of a given layer is provided to each node of the next layer. In some embodiments, the output layer 112 may apply the following nonlinear function, such as a Softmax function, to the output layer's input vector x, where the predicted probability for the P(y=j|x) for the jth class is determined from vector x and a weighting vector w:

$$P ( y = j \vert x ) = \frac{{e}^{{x}^{T} {w}_{j}}}{{\sum }_{k = 1}^{K} {e}^{{x}^{T} {w}_{k}}} \qquad Equation 2$$

In some embodiments, the output layer 112 may provide two outputs giving probabilities of error in flow space, wherein the first provides the probability of the base call being correct, and the other provides the probability of the base call being incorrect.

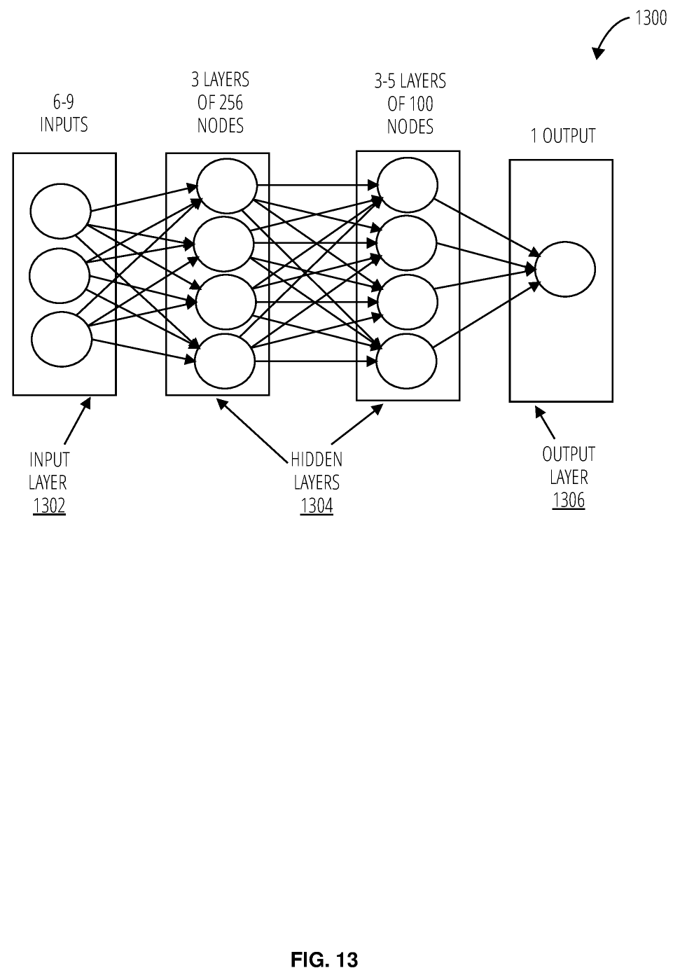

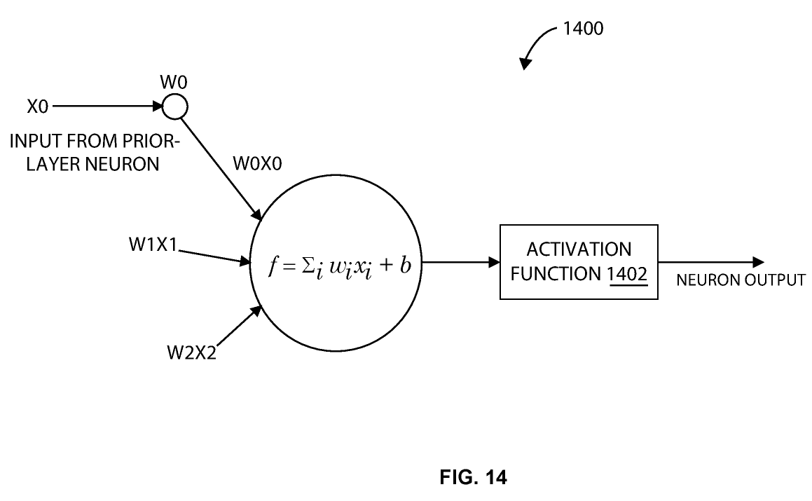

The neural network model may be a multilayer perceptron as depicted by the examples in FIG. 13 and FIG. 14. FIG. 13 illustrates an example of a basic deep neural network 1300. FIG. 14 illustrates an example of an artificial neuron 1400. In some embodiments, the weights and the bias of the neural network can be trained using measured feature vectors and base calls for a truth set of bases for a known nucleic acid sequence (labeled data set). For example, sequencing of E. coli with known DNA sequences can be used as known sequence of bases for the training set. In the training dataset, the probability of correct/incorrect calls can be calculated based on the ground truth of the base call. The training optimizes the weights of the processing nodes to apply to the input feature vectors. The methods for training comprise a machine learning algorithm, for example, a stochastic gradient descent (SGD) algorithm, RMSProp, Adam, Adadelta, Adagrad or other adaptive learning algorithm.

In some embodiments, the optimized weights and bias may be fixed after training. In subsequent runs, the fixed weights of the neural network model may be applied to feature vectors from nucleic acid sequencing runs to obtain the probability of error in flow space.

To estimate the distribution of probability of errors in flow space, a certain loss function may be used. The cross-entropy may provide a measure of similarity between two probability distributions, a predicted probability distribution P and a true probability distribution Q. For the true probability distribution Q,

Q(y=1)=y and Q(y=0)=1−y Equation 3

For the predicted distribution P,

P(y=1)=ŷ and P(y=0)=1−ŷ Equation 4

The cross-entropy for a measure of similarity between the probability distributions P and Q is given by,

H(Q,P)=ΣᵢQᵢ log(Pᵢ)=−y log ŷ−(1−y)log(1−ŷ) Equation 5

In some embodiments, the flow space quality Q_{f} may be calculated based on the predicted flow space probability of error, P_{f}, determined by the neural network model, as follows,

Q_{f}=−10 log(P_{f}) Equation 6

For a certain flow f and probability of error, assume the flow generates m base incorporations. So, the flow is measured to be an m-mer and its quality is predicted to be Q_{f}. The corresponding bases incorporated during the flow are {b₁, b₂, . . . , bₘ}, where b₁ is a first base incorporated, b₂ is a second base incorporated and bₘ is the m^{th} base incorporated for a homopolymer of length m. The probability of error in flow f can be found by:

P_{f}=1−P{f being recognized as m−mer}=Σ_{i=0,i≠m}^{N}P{f being recognized as i−mer} Equation 7

where m is the true length of the homopolymer. Empirically, from the ground-truth alignment with the reference sequence, the above distribution may be pre-calculated.

The base error probability P is given by

P{(n+1)th base is an error|(f being as m−mer)}=P{(f being recognized as i−mer)|(f being an m−mer)} Equation 8

where (n+1)th base is the next base incorporated after the n^{th} base of the same nucleotide in a given flow f.

Assuming independence between f being an m-mer and a base error, the probability of error in base bᵢ given the probability of error P_{b}_{ᵢ} in flow f is obtained as

P_{b}_{ᵢ}=P{(i+1)th base is an error}=P{(f being recognized as i−mer)|(f being an m−mer)}×P{(f being an m−mer)} Equation 9

where i<m.

The base quality value Q_{b}_{ᵢ} may be calculated as,

Q_{b}_{ᵢ}=−10 log(P_{b}_{ᵢ}) Equation 10

Using the above method, the flow space quality values are transformed into base quality values, or base quality scores. In some embodiments, the base quality value may be provided to the base caller 106. The base call may then be output with the base quality value for each reaction well. This process is performed for measurements from each well in the sequencer 102.

In some embodiments, an average of the base quality values for consecutive bases of a sequence of base calls over a window of previous bases, a current base and future bases may be calculated, where the window's position and size are configurable. The average base quality value may be provided to the base caller 106. The base caller 106 may compare the average base quality value with a threshold value. If the average base quality value is below the threshold value, the base caller 106 may cut the tail of the sequence after the current base and keep the portion of the sequence having higher quality. The threshold value may be set to a default value of 15, which equals −10 log(10^{-1.5}), or may be set by a user. The average base quality value may be calculated for a window of flows relative to the flow corresponding to the current base, where the window's position and size are configurable. The user may select and configure the window for base space or flow space. When the average base quality value is less than the threshold, the flow predictor parameters corresponding to subsequent flows will not be processed by the neural network to generate a probability of error. The averaging of the base quality values may be performed for each well in the sequencer 102.

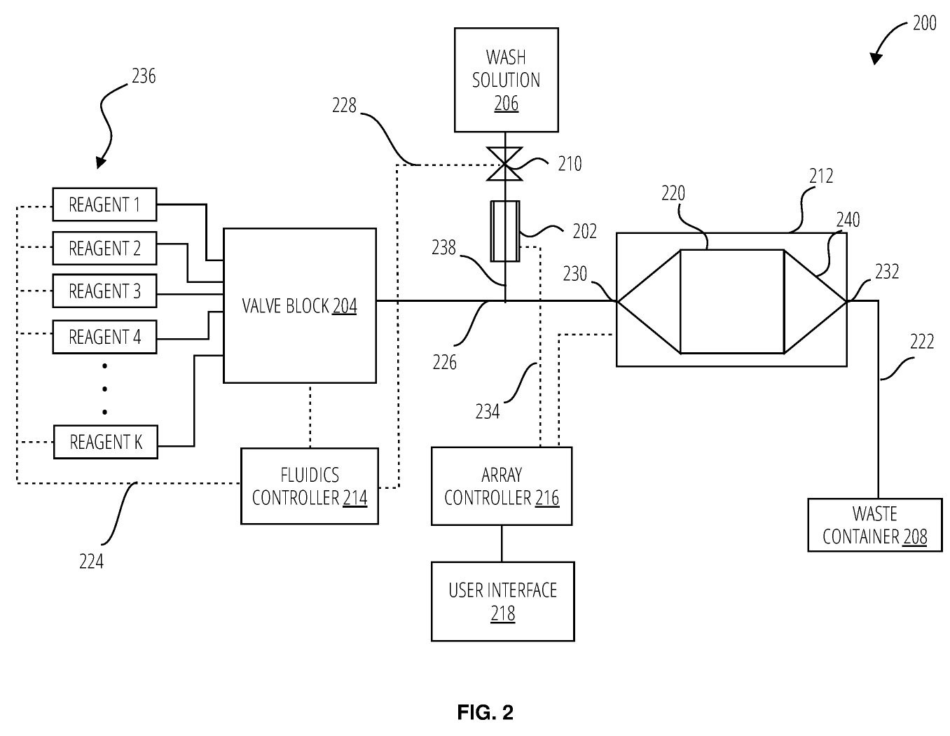

FIG. 2 illustrates components of a system for nucleic acid sequencing 200 according to an exemplary embodiment. The components include a flow cell and sensor array 212, a reference electrode 202, a plurality of reagents 236, a valve block 204, a wash solution 206, a valve 210, a fluidics controller 214, line 224, line 228, line 234, passage 222, passage 226, passage 238, a waste container 208, an array controller 216, and a user interface 218. The flow cell and sensor array 212 includes an inlet 230, an outlet 232, a microwell array 220, and a flow chamber 240 defining a flow path of reagents over the microwell array 220. The reference electrode 202 may be of any suitable type or shape, including a concentric cylinder with a fluid passage or a wire inserted into a lumen of passage 238. The reagents 236 may be driven through the fluid pathways, valves, and flow cell by pumps, gas pressure, or other suitable methods, and may be discarded into the waste container 208 after exiting the flow cell and sensor array 212. The fluidics controller 214 may control driving forces for the reagents 236 and the operation of valve 210 and valve block 204 with suitable software. The microwell array 220 may include an array of defined spaces or reaction confinement regions, such as microwells, for example, that is operationally associated with a sensor array so that, for example, each microwell has a sensor suitable for detecting an analyte or reaction property of interest. The microwell array 220 may preferably be integrated with the sensor array as a single device or chip. The flow cell may have a variety of designs for controlling the path and flow rate of reagents over the microwell array 220, and may be a microfluidics device. The array controller 216 may provide bias voltages and timing and control signals to the sensor, and collect and/or process output signals. The user interface 218 may display information from the flow cell and sensor array 212 as well as instrument settings and controls, and allow a user to enter or set instrument settings and controls.

In an exemplary embodiment, such a system may deliver reagents to the flow cell and sensor array 212 in a predetermined sequence, for predetermined durations, at predetermined flow rates, and may measure physical and/or chemical parameters providing information about the status of one or more reactions taking place in defined spaces or reaction confinement regions, such as, for example, microwells (or in the case of empty microwells, information about the physical and/or chemical environment therein). In an exemplary embodiment, the system may also control a temperature of the flow cell and sensor array 212 so that reactions take place and measurements are made at a known, and preferably, a predetermined temperature.

In an exemplary embodiment, such a system may be configured to let a single fluid or reagent contact the reference electrode 202 throughout an entire multi-step reaction. The valve 210 may be shut to prevent any wash solution 206 from flowing into passage 226 as the reagents are flowing. Although the flow of wash solution may be stopped, there may still be uninterrupted fluid and electrical communication between the reference electrode 202, passage 226, and the microwell array 220. The distance between the reference electrode 202 and the junction between passage 226 and passage 238 may be selected so that little or no amount of the reagents flowing in passage 226 and possibly diffusing into passage 238 reach the reference electrode 202. In an exemplary embodiment, the wash solution 206 may be selected as being in continuous contact with the reference electrode 202, which may be especially useful for multi-step reactions using frequent wash steps.

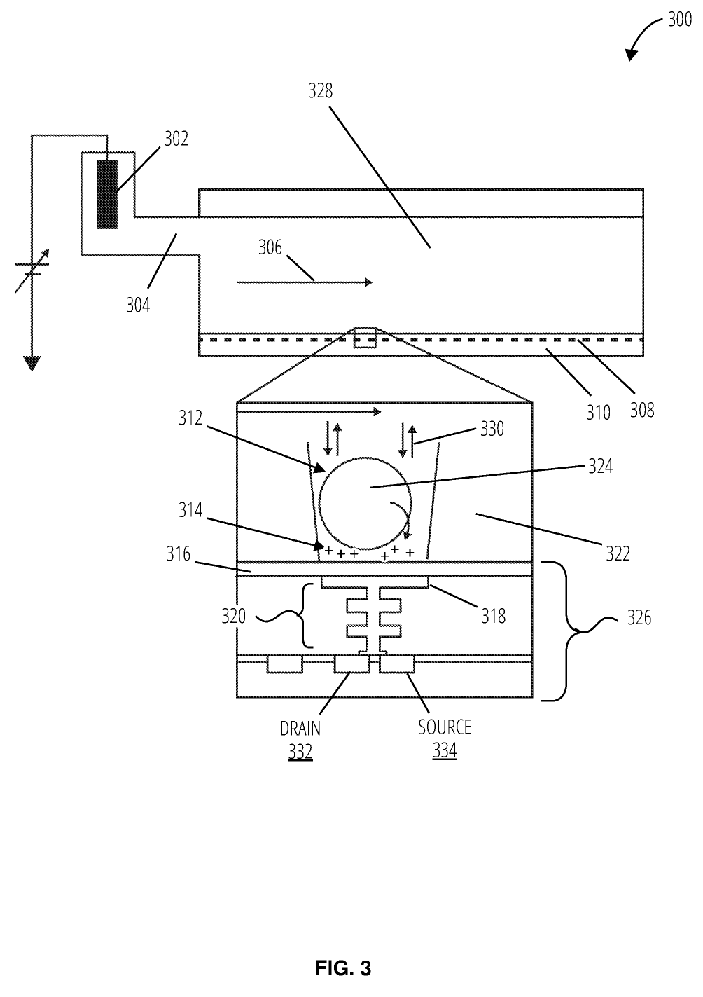

FIG. 3 illustrates cross-sectional and expanded view of a flow cell 300 for nucleic acid sequencing according to an exemplary embodiment. The flow cell 300 includes a microwell array 308, a sensor array 310, and a flow chamber 328 in which a reagent flow 306 may move across a surface of the microwell array 308, over open ends of microwells in the microwell array 308. A microwell 312 in the microwell array 308 may have any suitable volume, shape, and aspect ratio, which may be selected depending on one or more of any reagents, by-products, and labeling techniques used, and the microwell 312 may be formed in layer 322, for example, using any suitable microfabrication technique. A sensor 326 in the sensor array 310 may be an ion sensitive (ISFET) or a chemical sensitive (chemFET) sensor with a floating gate 320 having a sensor plate 318 separated from the microwell interior by a passivation layer 316, and may be predominantly responsive to (and generate an output signal related to) an amount of charge 314 present on the passivation layer 316 opposite of the sensor plate 318. Changes in the amount of charge 314 cause changes in the current between a source 334 and a drain 332 of the sensor 326, which may be used directly to provide a current-based output signal or indirectly with additional circuitry to provide a voltage output signal. Reactants, wash solutions, and other reagents may move into microwells primarily by diffusion 330. One or more analytical reactions to identify or determine characteristics or properties of an analyte of interest may be carried out in one or more microwells of the microwell array 308. Such reactions may generate directly or indirectly by-products that affect the amount of charge 314 adjacent to the sensor plate 318.

In some configurations, a reference electrode 302 may be fluidly connected to the flow chamber 328 via a flow passage 304. In some configurations, the microwell array 308 and the sensor array 310 may together form an integrated unit forming a bottom wall or floor of the flow cell 300. In some configurations, one or more copies of an analyte may be attached to a solid phase support 324, which may include microparticles, nanoparticles, beads, gels, and may be solid and porous, for example. The analyte may include a nucleic acid analyte, including a single copy and multiple copies, and may be made, for example, by rolling circle amplification (RCA), exponential RCA, or other suitable techniques to produce an amplicon without the need of a solid support.

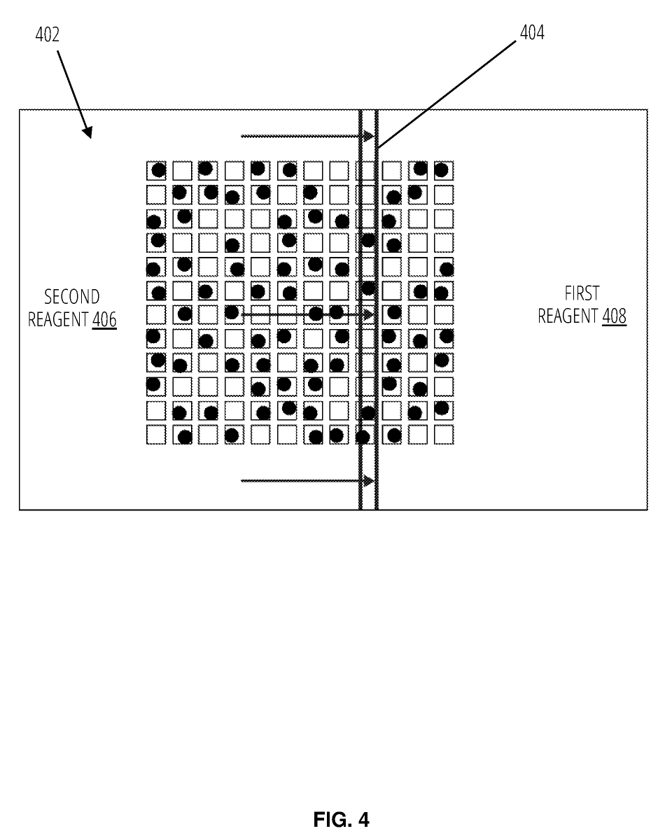

FIG. 4 illustrates a uniform flow front between successive reagents moving across a section 402 of a microwell array according to an exemplary embodiment. A “uniform flow front” between first reagent 408 and second reagent 406 generally refers to the reagents undergoing little or no mixing as they move, thereby keeping a boundary 404 between them narrow. The boundary may be linear for flow cells having inlets and outlets at opposite ends of their flow chambers, or it may be curvilinear for flow cells having central inlets (or outlets) and peripheral outlets (or inlets). In some configurations, the flow cell design and reagent flow rate may be selected so that each new reagent flow with a uniform flow front as it transits the flow chamber during a switch from one reagent to another.

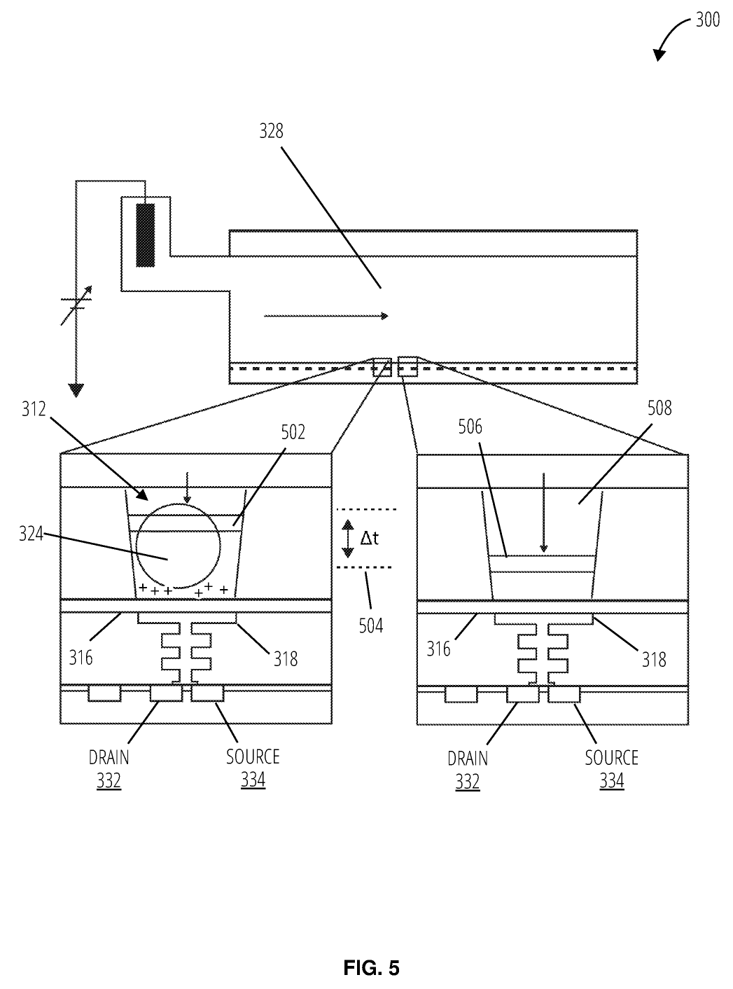

FIG. 5 illustrates a time delay associated with a diffusion of a reagent flow from a flow chamber 328 to a microwell 312 that contains an analyte and/or particle on a solid phase support 324 and to an empty microwell 508 according to an exemplary embodiment. The charging reagent flow may diffuse to the passivation layer 316 region opposite of the sensor plate 318. However, a diffusion front 502 of the reagent flow in the microwell 312 containing an analyte and/or particle on the solid phase support 324 is delayed relative to a diffusion front 506 of the reagent flow in the empty microwell 508, either because of a physical obstruction due to the analyte/particle or because of a buffering capacity of the analyte/particle.

In some configurations, a correlation between an observed time delay 504 in a change of output signal and the presence of an analyte/particle may be used to determine whether a microwell contains an analyte. To observe the time delay 504, the pH may be changed using a charging reagent from a first predetermined pH to a different pH, effectively exposing the sensors to a step-function change in pH that will produce a rapid change in charge on the sensor plates. The pH change between the first reagent and the charging reagent (which may sometimes be referred to herein as the “second reagent” or the “sensor-active” reagent) may be 2.0 pH units or less, 1.0 pH unit or less, 0.5 pH unit or less, or 0.1 pH unit or less, for example. The changes in pH may be made using conventional reagents, including HCl, NaOH, for example, at concentrations for DNA pH-based sequencing reactions in the range of from 5 to 200 μM, or from 10 to 100 μM, for example.

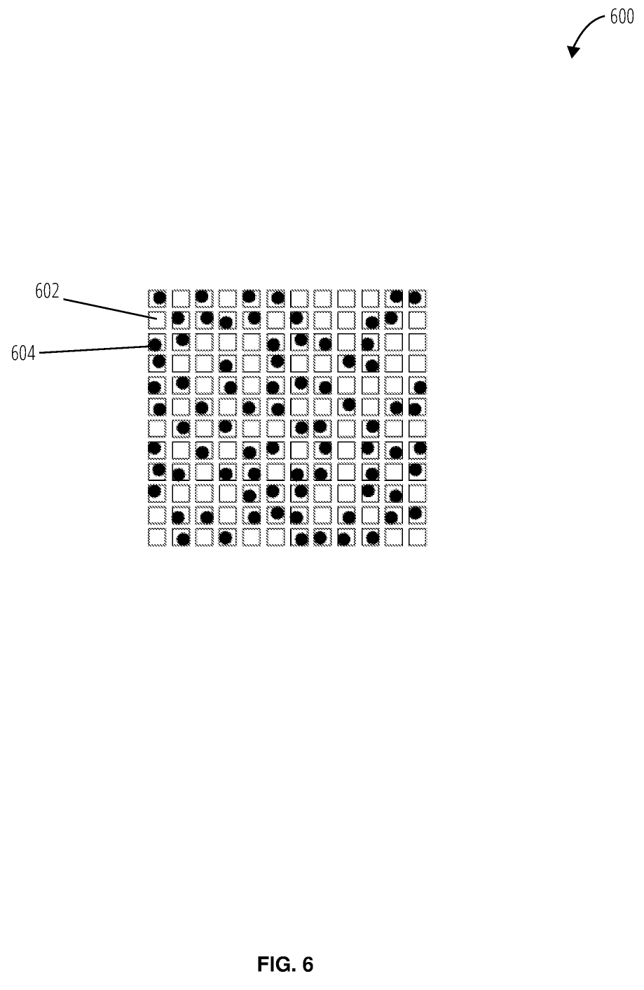

FIG. 6 illustrates an array section 600 including empty microwells 602 and analyte-containing microwells 604 according to an exemplary embodiment. The analytes may be randomly distributed among the microwells, and may include beads, for example.

In one embodiment, output signals collected from empty wells may be used to reduce or subtract noise in output signals collected from analyte-containing wells to improve a quality of such output signals. Such reduction or subtraction may be done using any suitable signal processing techniques. The noise component may be measured based on an average of output signals from multiple neighboring empty wells that may be in a vicinity of a well of interest, which may include weighted averages and functions of averages, for example, based on models of physical and chemical processes taking place in the wells.

In one embodiment, alternatively or in addition to neighboring empty wells, other sets of wells may be analyzed to characterize noise even better, which may include wells containing particles without an analyte, for example. The noise component or averages may be processed in various ways, including converting time domain functions of average empty well noise to frequency domain representations and using Fourier analysis to remove common noise components from output signals from non-empty wells.

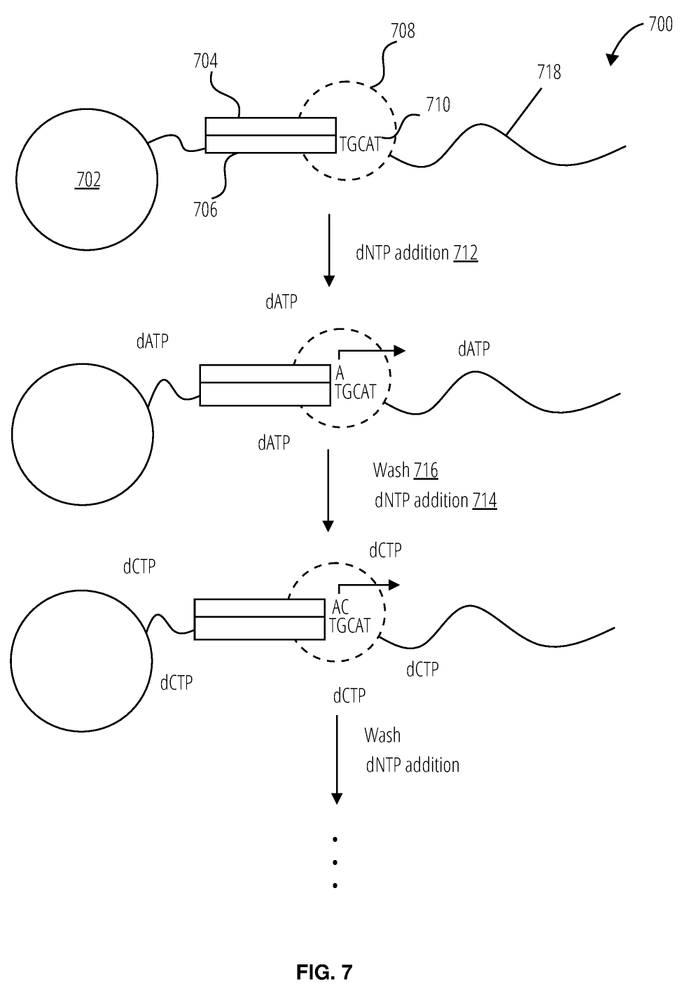

FIG. 7 illustrates schematically a process 700 for label-free, pH-based sequencing according to one embodiment. A template 718 with a primer binding site 706 are attached to a solid phase support 702. The template 718 may be attached as a clonal population to a solid support, such as a microparticle or bead, for example, and may be prepared as disclosed in U.S. Pat. No. 7,323,305, which is incorporated by reference herein in its entirety. A primer 704 and DNA polymerase 708 are operably bound to the template 718. As used herein, “operably bound” generally refers to a primer being annealed to a template so that the primer's 3′ end may be extended by a polymerase and that a polymerase is bound to such primer-template duplex (or in close proximity thereof) so that binding and/or extension may take place when dNTPs are added. In step 712, dNTP (shown as dATP) is added, and the DNA polymerase 708 incorporates a nucleotide “A” (since “T” is the next nucleotide in the template 718). In step 716, a wash is performed. In step 714, the next dNTP (shown as dCTP) is added, and the DNA polymerase 708 incorporates a nucleotide “C” (since “G” is the next nucleotide in the template 718). The pH-based nucleic acid sequencing, in which base incorporations may be determined by measuring hydrogen ions that are generated as natural by-products of polymerase-catalyzed extension reactions, may be performed using at least in part one or more features of Anderson et al., Sensors and Actuators B Chem., 129:79-86 (2008); Rothberg et al., U.S. Pat. Appl. Publ. No. 2009/0026082; and Pourmand et al., Proc. Natl. Acad. Sci., 103:6466-6470 (2006), which are all incorporated by reference herein in their entirety. In one embodiment, after each addition of a dNTP, an additional step may be performed in which the reaction chambers are treated with a dNTP-destroying agent, such as apyrase, to eliminate any residual dNTPs remaining in the chamber that might result in spurious extensions in subsequent cycles.

The output signals measured throughout this process depend on the number of nucleotide incorporations. Specifically, in each addition step, the polymerase extends the primer by incorporating added dNTP only if the next base in the template is complementary to the added dNTP. If there is one complementary base, there is one incorporation; if two, there are two incorporations; if three, there are three incorporations, and so on. With each incorporation, an hydrogen ion is released, and collectively a population released hydrogen ions change the local pH of the reaction chamber. The production of hydrogen ions is monotonically related to the number of contiguous complementary bases in the template (as well as to the total number of template molecules with primer and polymerase that participate in an extension reaction). Thus, when there is a number of contiguous identical complementary bases in the template (which may represent a homopolymer region), the number of hydrogen ions generated and thus the magnitude of the local pH change is proportional to the number of contiguous identical complementary bases (and the corresponding output signals are then sometimes referred to as “1-mer,” “2-mer,” “3-mer” output signals, etc.). If the next base in the template is not complementary to the added dNTP, then no incorporation occurs and no hydrogen ion is released (and the output signal is then sometimes referred to as a “O-mer” output signal). In each wash step of the cycle, an unbuffered wash solution at a predetermined pH may be used to remove the dNTP of the previous step in order to prevent misincorporations in later cycles. In one embodiment, the four different kinds of dNTP are added sequentially to the reaction chambers, so that each reaction is exposed to the four different dNTPs, one at a time. In one embodiment, the four different kinds of dNTP are added in the following sequence: dATP, dCTP, dGTP, dTTP, dATP, dCTP, dGTP, dTTP, etc., with each exposure followed by a wash step. Each exposure to a nucleotide followed by a washing step can be considered a “nucleotide flow.” Four consecutive nucleotide flows can be considered a “cycle.” For example, a two cycle nucleotide flow order can be represented by: dATP, dCTP, dGTP, dTTP, dATP, dCTP, dGTP, dTTP, with each exposure being followed by a wash step. Different flow orders are of course possible.

In one embodiment, template 718 may include a calibration sequence 710 that provides a known signal in response to the introduction of initial dNTPs. The calibration sequence 710 preferably contains at least one of each kind of nucleotide, may contain a homopolymer or may be non-homopolymeric, and may contain from 4 to 6 nucleotides in length, for example. In one embodiment, calibration sequence information from neighboring wells may be used to determine which neighboring wells contain templates capable of being extended (which may, in turn, allows identification of neighboring wells that may generate 0-mer signals, 1-mer signals, etc., in subsequent reaction cycles), and may be used to remove or subtract undesired noise components from output signals of interest.

In one embodiment, an average 0-mer signal may be modeled (which may be referred to herein as a “virtual 0-mer” signal) by taking into account (i) neighboring empty well output signals in a given cycle, and (ii) one or more effects of the presence of a particle and/or template on the shape of the reagent change noise curve (such as, e.g., the flattening and shifting in the positive time direction of an output signal of a particle-containing well relative to an output signal of an empty well. Such effects may be modeled to convert empty well output signals to virtual 0-mer output signals, which may in turn be used to subtract reagent change noise.

A sequence may be represented in “base-space” format (e.g., using a series or vector of nucleotide designations such as A, C, G, and T that correspond to the series of nucleotide species that were flowed and incorporated). A sequence may also be represented in “flow-space” format (e.g., using a series or vector of zeros and ones representing a non-incorporation event (a zero, “0”) for a given nucleotide flow or a nucleotide incorporation event (a one, “1”) for a given nucleotide flow). Thus, in flow-space format, the nucleotide flow order and whether and how many non-events and events occurred for any given nucleotide flow determine the flow-space format series of zeros and ones, which may be referred to as the flow order vector. (Of course, zeros and ones are merely convenient representations of a non-incorporation event and a nucleotide incorporation event, and any other symbol or designation could be used alternatively to represent and/or identify such non-events and events.) Also, in some exemplary embodiments, a homopolymer region may be represented by a whole number greater than one, rather than the respective number of one's in series (e.g., one might opt to represent a “T” flow resulting in an incorporation followed by an “A” flow resulting in two incorporations by “12” rather than “111” in flow-space).

To illustrate the interplay between base-space vectors, flow-space vectors, and nucleotide flow orders, one may consider, for example, an underlying template sequence beginning with “TA” subjected to multiple cycles of a nucleotide flow order of “TACG.” The first flow, “T,” would result in a non-incorporation because it is not complementary to the template's first base, “T.” In the base-space vector, no nucleotide designation would be inserted; in the flow-space vector, a “0” would be inserted, leading to “0.” The second flow, “A,” would result in an incorporation because it is complementary to the template's first base, “T.” In the base-space vector, an “A” would be inserted, leading to “A”; in the flow-space vector, a “1” would be inserted, leading to “01.” The third flow, “C,” would result in a non-incorporation because it is not complementary to the template's second base, “A.” In the base-space vector, no nucleotide designation would be inserted; in the flow-space vector, a “0” would be inserted, leading to “010.” The fourth flow, “G,” would result in a non-incorporation because it is not complementary to the template's second base, “A.” In the base-space vector, no nucleotide designation would be inserted; in the flow-space vector, a “0” would be inserted, leading to “0100.” The fifth flow, “T,” would result in an incorporation because it is complementary to the template's second base, “A.” In the base-space vector, a “T” would be inserted, leading to “AT”; in the flow-space vector, a “1” would be inserted, leading to “01001.” (Note: if the analysis were to contemplate a potentially longer template, an “X” could be inserted here instead because additional “A's” could potentially be present in the template in the case of a longer homopolymer, which would allow for more than one incorporation during the fifth flow, leading to “0100X.”) The base-space vector thus shows only the sequence of incorporated nucleotides, whereas the flow-space vector shows more expressly the incorporation status corresponding to each flow. Whereas a base-space representation may be fixed and remain common for various flow orders, the flow-based representation depends on the particular flow order. Knowing the nucleotide flow order, one can infer either vector from the other. Of course, the base-space vector could be represented using complementary bases rather than the incorporated bases.

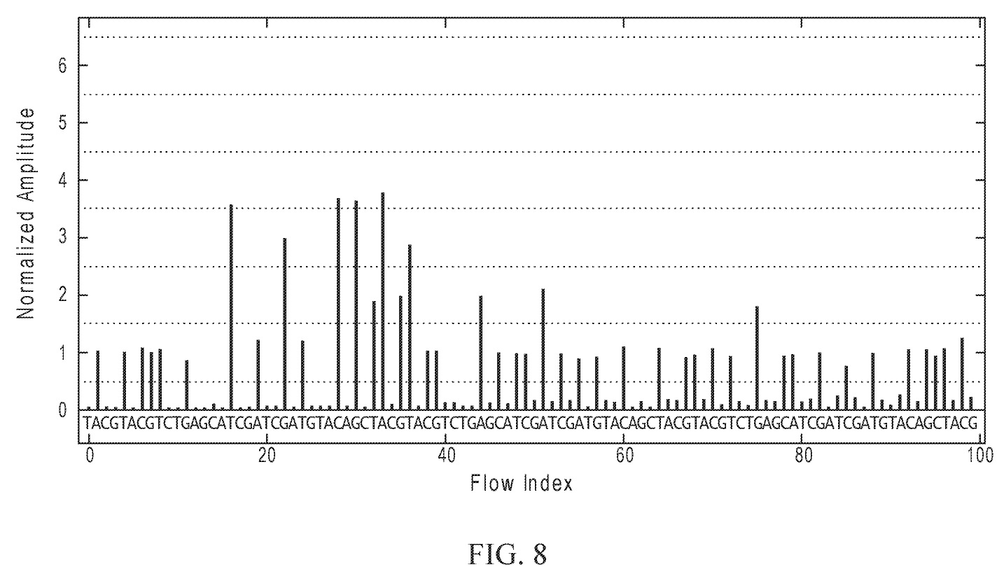

FIG. 8 shows an exemplary representation of flow space signal measurements from which base calls may be made. In this example, the x-axis shows the flow number and nucleotide that was flowed in a flow sequence. The bars in the graph show the amplitudes of the flow space signal measurements for each flow from a particular location of a microwell in the sensor array. The numerals on the y-axis show the corresponding number of nucleotide incorporations that may be estimated by rounding to the nearest integer, for example. The number of nucleotide incorporations indicates a homopolymer length. The flow space signal measurements may be raw acquisition data or data having been processed, such as, e.g., by scaling, background filtering, normalization, correction for signal decay, and/or correction for phase errors or effects, etc. The base calls may be made by analyzing any suitable signal characteristics (e.g., signal amplitude or intensity). The structure and/or design of sensor array, signal processing and base calling for use with the present teachings may include one or more features described in U.S. Pat. Appl. Publ. No. 2013/0090860, published Apr. 11, 2013, incorporated by reference herein in its entirety.

For example, given that a nucleotide flow order is:

ACTGACTGA

and the respective signals generated by a well after each nucleotide flow are:

0.1, 0.3, 0.2, 1.4, 0.3, 1.2, 0.8, 1.5, 0.7

Based on the nucleotide flow sequence, a putative nucleic acid sequence is generated using the signals rounded to the nearest integer (as either a nucleotide incorporation event occurred or did not occur, but not partially). Thus, the above nucleotide flow order and signals establish a putative nucleic acid sequence as follows:

| FLOW | SEQUENCE | BASE SEQUENCE |
|---|---|---|
| A | 0.1 | |
| C | 0.3 | |
| T | 0.2 | |
| G | 1.4 | → G |
| A | 0.3 | |
| C | 1.2 | → C |
| T | 0.8 | → T |
| G | 1.5 | → G |
| A | 0.7 | → A |

Once the base sequence for the sequence read is determined, the sequence read may be aligned to a reference sequence to form aligned sequence reads. Methods for forming aligned sequence reads for use with the present teachings may include one or more features described in U.S. Pat. Appl. Publ. No. 2012/0197623, published Aug. 2, 2012, incorporated by reference herein in its entirety.

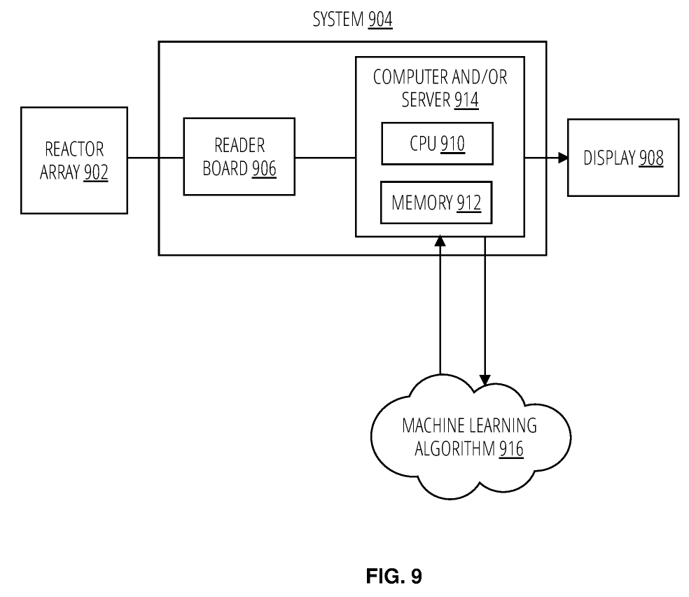

FIG. 9 illustrates a system 904 for nucleic acid sequencing according to one embodiment. The system includes a reactor array 902; a reader board 906; a computer and/or server 914, which includes a CPU 910 and a memory 912; and a display 908, which may be internal and/or external. The computer and/or server 914 may communicate information from processes involved in signal processing and base calling to a machine learning algorithm 916. The machine learning algorithm 916 may utilize the information provided by the these processes to improve the quality score prediction of the sequencing data. One or more of these components may be used to perform or implement one or more aspects of embodiments described herein.

In one embodiment, the signal processor 104 may be configured to perform or implement one or more of the teachings disclosed in Rearick et al., U.S. patent application Ser. No. 13/339,846, titled “Models for Analyzing Data From Sequencing-by-Synthesis Operations”, filed Dec. 29, 2011, and in Hubbell, U.S. patent application Ser. No. 13/339,753, titled “Time-Warped Background Signal for Sequencing-by-Synthesis Operations”, filed Dec. 29, 2011, which are all incorporated by reference herein in their entirety.

In one embodiment, the signal processor 104 may store, transmit, and/or output raw incorporation signals and related information and data in raw WELLS file format, for example. The signal processor may output a raw incorporation signal per defined space and per flow, for example.

In some configurations, a base caller 106 may be configured to transform a raw incorporation signal into a base call and compile consecutive base calls associated with a sample nucleic acid template into a read. A base call refers to a particular nucleotide identification (e.g., dATP (“A”), dCTP (“C”), dGTP (“G”), or dTTP (“T”)). The base caller 106 may perform one or more signal normalizations, signal phase and signal droop (e.g, enzyme efficiency loss) estimations, and signal corrections, and it may identify or estimate base calls for each flow for each defined space. The base caller 106 may share, transmit or output non-incorporation events as well as incorporation events.

In some configurations, the base caller 106 may be configured to perform or implement one or more of the teachings disclosed in Davey et al., U.S. patent application Ser. No. 13/283,320, filed Oct. 27, 2011, incorporated by reference herein in its entirety. In some configurations, the base caller 106 may receive data in WELLS file format. The base caller 106 may store, transmit, and/or output reads and related information in a standard flowgram format (“SFF”), for example.

FIG. 10 gives examples of flow predictor parameters that may be provided in a feature vector to the input layer 108 of the neural network. Flow space signal measurements are referred to as “flow values” in FIG. 10. The penalty residual parameter is a difference between a predicted flow space signal measurement and an actual flow space signal measurement. The local noise parameter is the maximum difference between the flow space signal measurements an integer in a +/−1 base range around the current base flow. Referring to FIG. 8, the difference is between the normalized amplitude and nearest integer on the y-axis and the +/− base range refers to the flow indices on the x-axis. The high residual events parameter is the number of flows in a 20-flow window around the flow containing the base that have high residuals, where the residual is the difference between the predicted and measured flow space signal measurements. The multiple incorporations parameter is the number of bases incorporated during the flow, or homopolymer length. The penalty miscall parameter is a measure of certainty of a sequence of bases determined by the basecalling process compared to alternative candidate sequences. The environment noise parameter is the maximum difference between the flow space signal measurements an integer in a +/−10 base range around the current base flow. This is similar to the local noise parameter with a larger base range. The additive correction parameter is an additive correction term applied to the flow space signal measurement in an adaptive normalization performed by a basecalling process. For example, an additive correction term β may be an offset correction. Normalized flow space signal measurements may then be calculated according to M_{normalized,i}=(Mᵢ−βᵢ)/α, where M_{normalized,i} is a normalized measurement at flow i, Mᵢ is the flow space signal measurement at flow i, βᵢ is an additive correction at flow i, and α is a multiplicative correction term. The multiplicative correction parameter is a multiplicative correction term applied to the flow space signal measurement in the basecalling process. For example, a normalized flow space signal measurement be calculated according to M_{normalized,i}=Mᵢ/α, where M_{normalized,i} is a normalized measurement a flow i, Mᵢ is the flow space signal measurement at flow i, and a is the multiplicative correction term. The state inphase parameter is an indicator of the in phase incorporations by the polymerase within the same well for a given flow. Several of these parameters as applied for basecalling are further described in U.S. Pat. Appl. Publ. No. 2013/0090860, published Apr. 11, 2013, which is incorporated by reference herein in its entirety.

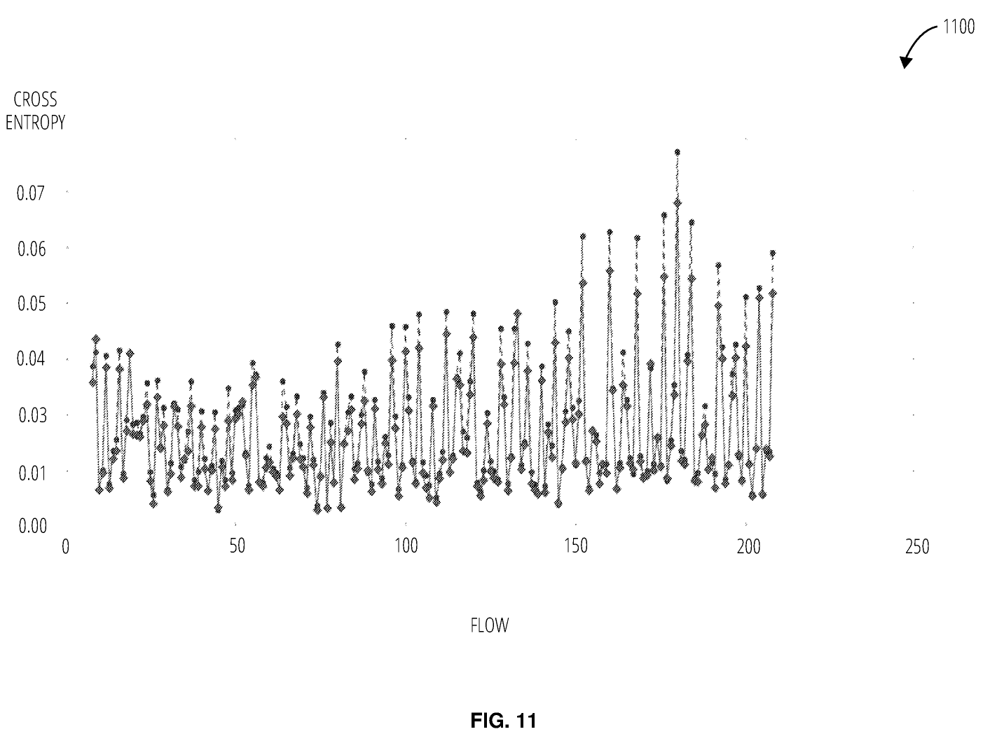

FIG. 11 shows plots of cross-entropy comparisons 1100 calculated for the neural network model. The x-axis depicts the flow number. The plot with the diamonds represents the cross-entropy values for the probability distribution resulting from the neural network model calculated with the true probability distribution. The plot with the circles represents the cross-entropy values for the probability distribution from the PHRED lookup table (as described by Brent Ewing, LaDeana W. Hillier, Michael C. Wendl, Phil Green; Base-calling of automated sequencer traces using Phred. I. accuracy assessment. Genome Research, Issue: 3, Volume: 8, Pages: 175-185. Feb. 28, 1998), calculated with the true probability distribution. The cross entropy values in FIG. 11 were calculated based on 1,000,000 reads. The lower cross-entropy values for probability distribution from the neural network model indicate greater similarity with the true probability distribution. The neural network model provides more accurate estimation of the flow space probability of error than the PHRED lookup table, as indicated by the lower cross-entropy.

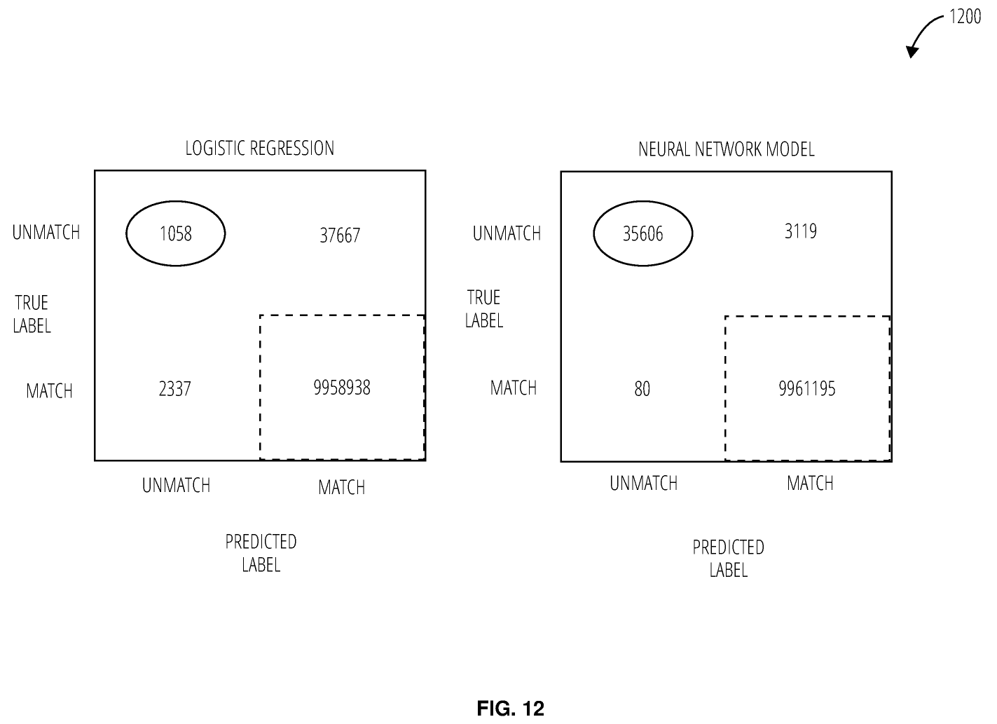

FIG. 12 depicts confusion matrices 1200 for the neural network model results versus logistic regression results. The ability to predict errors in the base calls are plotted for the neural network model and the logistic regression model. The upper left quadrant indicates error is predicted given that there is true error, the upper right quadrant indicates no error is predicted given that there is true error, the lower left quadrant indicates error is predicted given that there is no true error and the lower right quadrant indicates no error is predicted given that there is no true error. Higher numbers in the upper left and lower right quadrants indicate more accurate predictions. Logistic regression (left box) and the neural network model (right box) were applied to flow space data obtained from 10 million flows to predict error or no error in the base calls. The results show that the neural network model resulted in more accurate predictions of error or no error in the base calls.

FIG. 13 illustrates an example of a basic deep neural network 1300. A basic deep neural network 1300 is based on a collection of connected units or nodes called artificial neurons which loosely model the neurons in a biological brain. Each connection, like the synapses in a biological brain, can transmit a signal from one artificial neuron to another. An artificial neuron that receives a signal can process it and then signal additional artificial neurons connected to it.

In common implementations, the signal at a connection between artificial neurons is a real number, and the output of each artificial neuron is computed by some non-linear function (the activation function) of the sum of its inputs. The connections between artificial neurons are called ‘edges’ or axons. Artificial neurons and edges typically have a weight that adjusts as learning proceeds. The weight increases or decreases the strength of the signal at a connection. Artificial neurons may have a threshold (trigger threshold) such that the signal is only sent if the aggregate signal crosses that threshold. Typically, artificial neurons are aggregated into layers. Different layers may perform different kinds of transformations on their inputs. Signals travel from the first layer (the input layer 1302), to the last layer (the output layer 1306), possibly after traversing one or more intermediate layers, called hidden layers 1304.

In one embodiment, the basic deep neural network 1300 has an input layer 1302, six hidden layers 1304, and an output layer 1306. In other embodiments, there may be seven or eight hidden layers 1304. The input layer 1302 may receive six to nine input parameters. These are selected from the flow predictor parameters 1000. Each input is for one flow for one well. The basic deep neural network 1300 may then receive other inputs for different wells or another flow for the same well. The hidden layers 1304 may comprise two groups. The first group is connected to the input layer 1302 and comprises three layers, each with 256 nodes. These are fully connected to the previous and subsequent layer. The next group comprises 3-5 layers of 100 nodes, which are fully connected to the previous and subsequent layers. The numbers of layers and nodes per layer given in FIG. 13 are exemplary dimensions. In some embodiments, the neural network 1300 may be configured to have different number of layers and nodes per layer. The output layer 1306 comprises one node, which is the value for the probability of error for that flow for the well. The output layer 1306 may have a Softmax function performed on the output. The probability of error for the flow f may then be transformed, as described with respect to FIG. 1, to generate a base quality value.

Referring to FIG. 14, an artificial neuron 1400 receiving inputs from predecessor neurons comprises the following components:
inputs xᵢ;
weights wᵢ applied to the inputs;
an optional threshold (b); and
an activation function 1402, such as ReLU or sigmoid function, that computes the output from the previous neuron inputs and threshold, if any.

An input neuron has no predecessor but serves as input interface for the whole network. Similarly an output neuron has no successor and thus serves as output interface of the whole network.

The network includes connections, each connection transferring the output of a neuron in one layer to the input of a neuron in a next layer. Each connection carries an input x and is assigned a weight w.

The activation function 1402 may be applied to a sum of products of the weighted values of the inputs of the predecessor neurons.

The learning rule is a rule or an algorithm which modifies the parameters of the neural network, in order for a given input to the network to produce a favored output. This learning process typically involves modifying the weights and thresholds of the neurons and connections within the network.

In one embodiment, the hidden layers 1304 utilize a sigmoid activation function 1402, such as depicted in equation 1 above. The output layer 1306 may utilize a Softmax function.

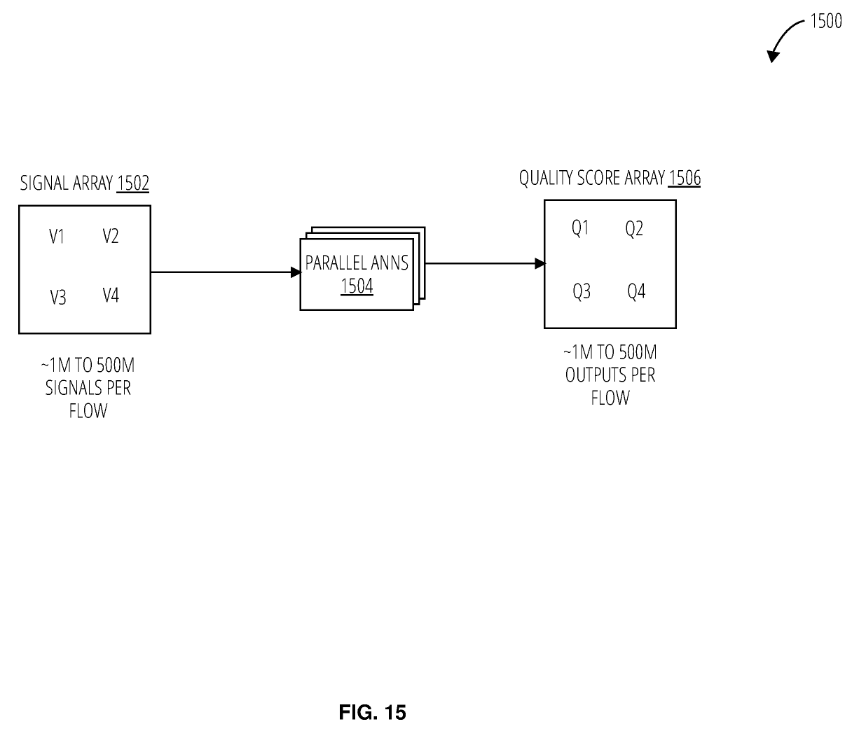

Referring to FIG. 15, a quality score system 1500 comprises a signal array 1502, parallel artificial neural networks (ANNs) 1504, and a quality score array 1506. The signal array 1502 comprises a vector of flow predictor parameters for each active well per flow (depicted as V1-V4, for a four well system). Each vector of flow predictor parameters is then sent to one of the parallel artificial neural networks 1504. Here, four parallel neural networks 1504 are utilized, one for each vector. Each of the parallel artificial neural networks 1504 generates an output probability of error for the input. The output probability of error is then sent to the quality score array 1506, which may then be converted, as described with respect to FIG. 1, into an array of base quality scores (depicted as Q1-Q4 for the four well system). This process may then be repeated for each flow. As the average base quality score of a certain well calculated over a window of flows may be below a threshold value, the flow predictor parameters from subsequent flows for this well may not be processed. For example, an average value of consecutive Q2 quality scores for a window from neighboring bases of the current flow may be below the threshold value. For subsequent flows, the vector V2 may be trimmed and three parallel artificial neural networks 1504 may be utilized instead of four parallel artificial neural networks 1504.

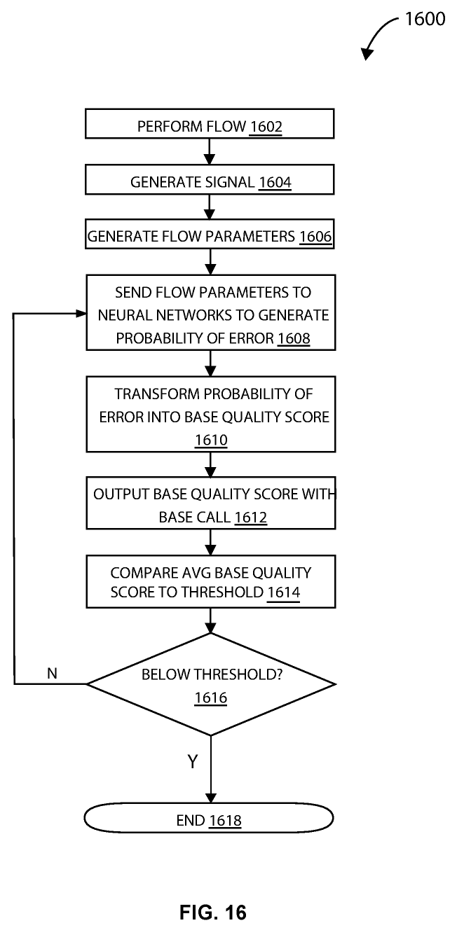

Referring to FIG. 16, a method 1600 performs a flow on a well (block 1602). The method 1600 may operate on multiple wells simultaneously. A signal is generated (block 1604). The signal may be proportional to the number of bases incorporated during a flow. Flow predictor parameters are then generated (block 1606). Exemplary flow predictor parameters are depicted in FIG. 10. The flow predictor parameters are sent to the neural network to generate a probability of error (block 1608). For multiple wells, the quality score system 1500 may be utilized. The probability of error is then transformed into a base quality score (block 1610). The base quality score is then output with the base call (block 1612). The method 1600 may calculate the average quality scores over a window of previous bases, a current base and future bases, where the window's size and position are configurable. The average base quality score is then compared to a threshold value (block 1614). The method 1600 determines whether the base quality score is below the threshold value (decision block 1616). If so, the method 1600 ends (done block 1618) and the basecalling of this particular well ends. If not, the method 1600 is performed for the next flow for the particular well beginning at block 1608.

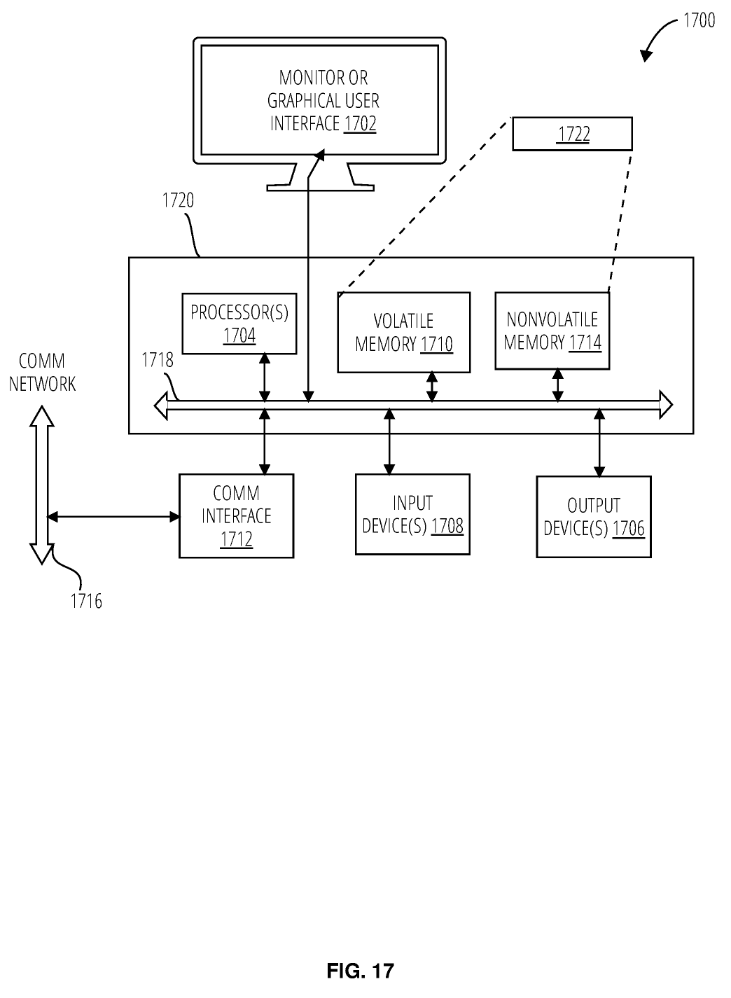

FIG. 17 is an example block diagram of a computing device 1700 that may incorporate embodiments of the present invention. FIG. 17 is merely illustrative of a machine system to carry out aspects of the technical processes described herein, and does not limit the scope of the claims. One of ordinary skill in the art would recognize other variations, modifications, and alternatives. In one embodiment, the computing device 1700 typically includes a monitor or graphical user interface 1702, a data processing system 1720, a communication network interface 1712, input device(s) 1708, output device(s) 1706, and the like.

As depicted in FIG. 17, the data processing system 1720 may include one or more processor(s) 1704 that communicate with a number of peripheral devices via a bus subsystem 1718. These peripheral devices may include input device(s) 1708, output device(s) 1706, communication network interface 1712, and a storage subsystem, such as a volatile memory 1710 and a nonvolatile memory 1714.

The volatile memory 1710 and/or the nonvolatile memory 1714 may store computer-executable instructions and thus forming logic 1722 that when applied to and executed by the processor(s) 1704 implement embodiments of the processes and neural networks disclosed herein.

The input device(s) 1708 include devices and mechanisms for inputting information to the data processing system 1720. These may include a keyboard, a keypad, a touch screen incorporated into the monitor or graphical user interface 1702, audio input devices such as voice recognition systems, microphones, and other types of input devices. In various embodiments, the input device(s) 1708 may be embodied as a computer mouse, a trackball, a track pad, a joystick, wireless remote, drawing tablet, voice command system, eye tracking system, and the like. The input device(s) 1708 typically allow a user to select objects, icons, control areas, text and the like that appear on the monitor or graphical user interface 1702 via a command such as a click of a button or the like.

The output device(s) 1706 include devices and mechanisms for outputting information from the data processing system 1720. These may include the monitor or graphical user interface 1702, speakers, printers, infrared LEDs, and so on as well understood in the art.

The communication network interface 1712 provides an interface to communication networks (e.g., communication network 1716) and devices external to the data processing system 1720. The communication network interface 1712 may serve as an interface for receiving data from and transmitting data to other systems. Embodiments of the communication network interface 1712 may include an Ethernet interface, a modem (telephone, satellite, cable, ISDN), (asynchronous) digital subscriber line (DSL), FireWire, USB, a wireless communication interface such as BlueTooth or WiFi, a near field communication wireless interface, a cellular interface, and the like.

The communication network interface 1712 may be coupled to the communication network 1716 via an antenna, a cable, or the like. In some embodiments, the communication network interface 1712 may be physically integrated on a circuit board of the data processing system 1720, or in some cases may be implemented in software or firmware, such as “soft modems”, or the like.

The computing device 1700 may include logic that enables communications over a network using protocols such as HTTP, TCP/IP, RTP/RTSP, IPX, UDP and the like.

The volatile memory 1710 and the nonvolatile memory 1714 are examples of tangible media configured to store computer readable data and instructions to implement various embodiments of the processes described herein. Other types of tangible media include removable memory (e.g., pluggable USB memory devices, mobile device SIM cards), semiconductor memories such as flash memories, non-transitory read-only-memories (ROMS), battery-backed volatile memories, networked storage devices, and the like. The volatile memory 1710 and the nonvolatile memory 1714 may be configured to store the basic programming and data constructs that provide the functionality of the disclosed processes and other embodiments thereof that fall within the scope of the present invention.

Logic 1722 that implements embodiments of the present invention may be embodied by the volatile memory 1710 and/or the nonvolatile memory 1714. Instructions of said logic 1722 may be read from the volatile memory 1710 and/or nonvolatile memory 1714 and executed by the processor(s) 1704. The volatile memory 1710 and the nonvolatile memory 1714 may also provide a repository for storing data used by the logic 1722.

The volatile memory 1710 and the nonvolatile memory 1714 may include a number of memories including a main random access memory (RAM) for storage of instructions and data during program execution and a read only memory (ROM) in which read-only non-transitory instructions are stored. The volatile memory 1710 and the nonvolatile memory 1714 may include a file storage subsystem providing persistent (non-volatile) storage for program and data files. The volatile memory 1710 and the nonvolatile memory 1714 may include removable storage systems, such as removable flash memory.

The bus subsystem 1718 provides a mechanism for enabling the various components and subsystems of data processing system 1720 communicate with each other as intended. Although the communication network interface 1712 is depicted schematically as a single bus, some embodiments of the bus subsystem 1718 may utilize multiple distinct busses.

It will be readily apparent to one of ordinary skill in the art that the computing device 1700 may be a device such as a smartphone, a desktop computer, a laptop computer, a rack-mounted computer system, a computer server, or a tablet computer device. As commonly known in the art, the computing device 1700 may be implemented as a collection of multiple networked computing devices. Further, the computing device 1700 will typically include operating system logic (not illustrated) the types and nature of which are well known in the art.

The structure and/or design of sensor array, signal processing and base calling for use with the present teachings may include one or more features described in U.S. Pat. Appl. Publ. No. 2012/0173159, published Jul. 5, 2012, incorporated by reference herein in its entirety.

Terms used herein should be accorded their ordinary meaning in the relevant arts, or the meaning indicated by their use in context, but if an express definition is provided, that meaning controls.

“ReLU” in this context refers to a rectifier function, an activation function defined as the positive part of its input. It is also known as a ramp function and is analogous to half-wave rectification in electrical signal theory. ReLU is a popular activation function in deep neural networks.

“Sigmoid function” in this context refers to a function of the form f(x)=1/(exp(−x)). The sigmoid function is used as an activation function in artificial neural networks. It has the property of mapping a wide range of input values to the range 0-1, or sometimes −1 to 1.

“Loss function” in this context, also referred to as the cost function or error function (not to be confused with the Gauss error function), is a function that maps values of one or more variables onto a real number intuitively representing some “cost” associated with those values.

“Softmax function” in this context refers to a function of the form f(xᵢ)=exp(x₁)/sum(exp(xᵢ)) where the sum is taken over a set of x. Softmax is used at different layers (often at the output layer) of artificial neural networks to predict classifications for inputs to those layers. The Softmax function calculates the probabilities distribution of the event xᵢ over ‘n’ different events. In general sense, this function calculates the probabilities of each target class over all possible target classes. The calculated probabilities are helpful for predicting that the target class is represented in the inputs. The main advantage of using Softmax is the output probabilities range. The range will extend from 0 to 1, and the sum of all the probabilities will be equal to one. If the Softmax function used for multi-classification model it returns the probabilities of each class and the target class will have the high probability. The formula computes the exponential (e-power) of the given input value and the sum of exponential values of all the values in the inputs. Then the ratio of the exponential of the input value and the sum of exponential values is the output of the Softmax function.

“Backpropagation” in this context refers to an algorithm used in artificial neural networks to calculate a gradient that is needed in the calculation of the weights to be used in the network. It is commonly used to train deep neural networks, a term referring to neural networks with more than one hidden layer. For backpropagation, the loss function calculates the difference between the network output and its expected output, after a case propagates through the network.

“Base caller” in this context refers to an algorithm that determines the bases of a sequence during analysis.

“Basecalling” in this context refers to a process that identifies each base in the sample and the order in which the bases are arranged and marks locations where there is some question about the base identification, such as when two bases seem to occur at the same position, with an N (instead of one of the four bases A, C, G, and T).

“Circuitry” in this context refers to electrical circuitry having at least one discrete electrical circuit, electrical circuitry having at least one integrated circuit, electrical circuitry having at least one application specific integrated circuit, circuitry forming a general purpose computing device configured by a computer program (e.g., a general purpose computer configured by a computer program which at least partially carries out processes or devices described herein, or a microprocessor configured by a computer program which at least partially carries out processes or devices described herein), circuitry forming a memory device (e.g., forms of random access memory), or circuitry forming a communications device (e.g., a modem, communications switch, or optical-electrical equipment).

“Firmware” in this context refers to software logic embodied as processor-executable instructions stored in read-only memories or media.

“Hardware” in this context refers to logic embodied as analog or digital circuitry.

“Logic” in this context refers to machine memory circuits, non-transitory machine readable media, and/or circuitry which by way of its material and/or material-energy configuration comprises control and/or procedural signals, and/or settings and values (such as resistance, impedance, capacitance, inductance, current/voltage ratings, etc.), that may be applied to influence the operation of a device. Magnetic media, electronic circuits, electrical and optical memory (both volatile and nonvolatile), and firmware are examples of logic. Logic specifically excludes pure signals or software per se (however does not exclude machine memories comprising software and thereby forming configurations of matter).

“Software” in this context refers to logic implemented as processor-executable instructions in a machine memory (e.g. read/write volatile or nonvolatile memory or media).

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

Various logic functional operations described herein may be implemented in logic that is referred to using a noun or noun phrase reflecting said operation or function. For example, an association operation may be carried out by an “associator” or “correlator”. Likewise, switching may be carried out by a “switch”, selection by a “selector”, and so on.

According to an exemplary embodiment, there is provided a method for estimating quality values of nucleotide base calls, comprising: (a) receiving flow space signal measurements from a reaction confinement region, the flow space signal measurements generated in response to a nucleotide flow to the reaction confinement region in an array of reaction confinement regions; (b) generating a base call and a plurality of flow predictor features corresponding to the nucleotide flow based on the flow space signal measurements; (c) applying an artificial neural network to the plurality of flow predictor features to generate a flow space probability of error; and (d) determining a base quality value based on the flow space probability of error. The step of determining the base quality value may be calculated by multiplying (−10) times the log of the flow space probability of error. The method may further include averaging a number of base quality values corresponding to a number of consecutive bases in a sequence of base calls to form an average base quality value. The step of generating a base call and a plurality of flow predictor features may be terminated when the average base quality value is less than a threshold. The step of applying an artificial neural network may further comprise applying a plurality of parallel neural networks, wherein a given neural network of the plurality of parallel neural networks is applied to the plurality of flow predictor features corresponding to a given reaction confinement region in the array of reaction confinement regions to provide the flow space probability of error corresponding to the given reaction confinement region. For the parallel neural networks, the step of determining a base quality value based on the flow space probability of error provides an array of base quality values corresponding to the array of reaction confinement regions. The method may further comprise training the artificial neural network by sequencing an E. coli sample having a known sequence of bases, wherein the sequencing provides a training set of flow space signal measurements for the step of receiving. The training may further comprise adjusting weights of the artificial neural network using a machine learning algorithm.

According to an exemplary embodiment, there is provided a system for estimating quality values of nucleotide base calls, comprising a machine-readable memory and a processor configured to execute machine-readable instructions, which, when executed by the processor, cause the system to perform a method for compressing molecular tagged nucleic acid sequence data, comprising: (a) receiving, at the processor, flow space signal measurements from a reaction confinement region, the flow space signal measurements generated in response to a nucleotide flow to the reaction confinement region in an array of reaction confinement regions; (b) generating a base call and a plurality of flow predictor features corresponding to the nucleotide flow based on the flow space signal measurements; (c) applying an artificial neural network to the plurality of flow predictor features to generate a flow space probability of error; and (d) determining a base quality value based on the flow space probability of error. The step of determining the base quality value may be calculated by multiplying (−10) times the log of the flow space probability of error. The method may further include averaging a number of base quality values corresponding to a number of consecutive bases in a sequence of base calls to form an average base quality value. The step of generating a base call and a plurality of flow predictor features may be terminated when the average base quality value is less than a threshold. The step of applying an artificial neural network may further comprise applying a plurality of parallel neural networks, wherein a given neural network of the plurality of parallel neural networks is applied to the plurality of flow predictor features corresponding to a given reaction confinement region in the array of reaction confinement regions to provide the flow space probability of error corresponding to the given reaction confinement region. For the parallel neural networks, the step of determining a base quality value based on the flow space probability of error provides an array of base quality values corresponding to the array of reaction confinement regions. The method may further comprise training the artificial neural network by sequencing an E. coli sample having a known sequence of bases, wherein the sequencing provides a training set of flow space signal measurements for the step of receiving. The training may further comprise adjusting weights of the artificial neural network using a machine learning algorithm.

According to an exemplary embodiment, there is provided a non-transitory machine-readable storage medium comprising instructions which, when executed by a processor, cause the processor to perform a method for estimating quality values of nucleotide base calls, comprising: (a) receiving, at the processor, flow space signal measurements from a reaction confinement region, the flow space signal measurements generated in response to a nucleotide flow to the reaction confinement region in an array of reaction confinement regions; (b) generating a base call and a plurality of flow predictor features corresponding to the nucleotide flow based on the flow space signal measurements; (c) applying an artificial neural network to the plurality of flow predictor features to generate a flow space probability of error; and (d) determining a base quality value based on the flow space probability of error. The step of determining the base quality value may be calculated by multiplying (−10) times the log of the flow space probability of error. The method may further include averaging a number of base quality values corresponding to a number of consecutive bases in a sequence of base calls to form an average base quality value. The step of generating a base call and a plurality of flow predictor features may be terminated when the average base quality value is less than a threshold. The step of applying an artificial neural network may further comprise applying a plurality of parallel neural networks, wherein a given neural network of the plurality of parallel neural networks is applied to the plurality of flow predictor features corresponding to a given reaction confinement region in the array of reaction confinement regions to provide the flow space probability of error corresponding to the given reaction confinement region. For the parallel neural networks, the step of determining a base quality value based on the flow space probability of error provides an array of base quality values corresponding to the array of reaction confinement regions. The method may further comprise training the artificial neural network by sequencing an E. coli sample having a known sequence of bases, wherein the sequencing provides a training set of flow space signal measurements for the step of receiving. The training may further comprise adjusting weights of the artificial neural network using a machine learning algorithm.

What is claimed is:

1. A method for estimating quality values of nucleotide base calls, comprising:
receiving flow space signal measurements from a reaction confinement region, the flow space signal measurements generated in response to a nucleotide flow to the reaction confinement region in an array of reaction confinement regions;
generating a base call and a plurality of flow predictor features corresponding to the nucleotide flow based on the flow space signal measurements;
applying an artificial neural network to the plurality of flow predictor features to generate a flow space probability of error; and
determining a base quality value based on the flow space probability of error.

2. The method of claim 1, wherein determining the base quality value is calculated by multiplying (−10) times a log of the flow space probability of error.

3. The method of claim 1, further comprising averaging a number of base quality values corresponding to a number of consecutive bases in a sequence of base calls to form an average base quality value.

4. The method of claim 3, wherein the step of generating a base call and a plurality of flow predictor features is terminated when the average base quality value is less than a threshold.

5. The method of claim 1, wherein the applying an artificial neural network further comprises applying a plurality of parallel neural networks, wherein a given neural network of the plurality of parallel neural networks is applied to the plurality of flow predictor features corresponding to a given reaction confinement region in the array of reaction confinement regions to provide the flow space probability of error corresponding to the given reaction confinement region.

6. The method of claim 5, wherein the determining a base quality value based on the flow space probability of error provides an array of base quality values corresponding to the array of reaction confinement regions.

7. The method of claim 1, further comprising training the artificial neural network by sequencing an E. coli sample having a known sequence of bases, wherein the sequencing provides a training set of flow space signal measurements for the step of receiving.

8. The method of claim 7, wherein the training further comprises adjusting weights of the artificial neural network using a machine learning algorithm.

9. A system for estimating quality values of nucleotide base calls, comprising:
a machine-readable memory; and
a processor configured to execute machine-readable instructions, which, when executed by the processor, cause the system to perform a method, comprising:
receiving, at the processor, flow space signal measurements from a reaction confinement region, the flow space signal measurements generated in response to a nucleotide flow to the reaction confinement region in an array of reaction confinement regions;
generating a base call and a plurality of flow predictor features corresponding to the nucleotide flow based on the flow space signal measurements;
applying an artificial neural network to the plurality of flow predictor features to generate a flow space probability of error; and
determining a base quality value based on the flow space probability of error.

10. The system of claim 9, wherein the determining the base quality value is calculated by multiplying (−10) times a log of the flow space probability of error.

11. The system of claim 9, wherein the method further comprises averaging a number of base quality values corresponding to a number of consecutive bases in a sequence of base calls to form an average base quality value.

12. The system of claim 11, wherein the step of generating a base call and a plurality of flow predictor features is terminated when the average base quality value is less than a threshold.

13. The system of claim 9, wherein the applying an artificial neural network further comprises applying a plurality of parallel neural networks, wherein a given neural network of the plurality of parallel neural networks is applied to the plurality of flow predictor features corresponding to a given reaction confinement region in the array of reaction confinement regions to provide the flow space probability of error corresponding to the given reaction confinement region.

14. The system of claim 13, wherein the determining a base quality value based on the flow space probability of error provides an array of base quality values corresponding to the array of reaction confinement regions.

15. The system of claim 9, wherein the method further comprises training the artificial neural network by sequencing an E. coli sample having a known sequence of bases, wherein the sequencing provides a training set of flow space signal measurements for the step of receiving.

16. The system of claim 15, wherein the training further comprises adjusting weights of the artificial neural network using a machine learning algorithm.

17. A non-transitory machine-readable storage medium comprising instructions which, when executed by a processor, cause the processor to perform a method for estimating quality values of nucleotide base calls, comprising:
receiving, at the processor, flow space signal measurements from a reaction confinement region, the flow space signal measurements generated in response to a nucleotide flow to the reaction confinement region in an array of reaction confinement regions;
generating a base call and a plurality of flow predictor features corresponding to the nucleotide flow based on the flow space signal measurements;
applying an artificial neural network to the plurality of flow predictor features to generate a flow space probability of error; and
determining a base quality value based on the flow space probability of error.

18. The non-transitory machine-readable storage medium of claim 17, further comprising instructions which cause the processor to perform the method, wherein the applying an artificial neural network further comprises applying a plurality of parallel neural networks, wherein a given neural network of the plurality of parallel neural networks is applied to the plurality of flow predictor features corresponding to a given reaction confinement region in the array of reaction confinement regions to provide the flow space probability of error corresponding to the given reaction confinement region.

19. The non-transitory machine-readable storage medium of claim 17, further comprising instructions which cause the processor to perform the method, further comprising training the artificial neural network by sequencing an E. coli sample having a known sequence of bases, wherein the sequencing provides a training set of flow space signal measurements for the step of receiving.

20. The non-transitory machine-readable storage medium of claim 19, further comprising instructions which cause the processor to perform the method, further comprising adjusting weights of the artificial neural network using a machine learning algorithm.
